(19) United States
(12) Patent Application Publication
(10) Pub. No.: US 2010/0081855 A1
(43) Pub. Date: Apr. 1, 2010

(54) Semi-Supported Dehydrogenation Catalyst

(75) Inventors: Joseph E. Pelati, Houston, TX (US); Hollie Craig, Seabrook, TX (US)

Correspondence Address: FINA TECHNOLOGY INC, PO BOX 674412, HOUSTON, TX, 77267-4412, US

(73) Assignee: Fina Technology, Inc., Houston, TX (US)

(21) Appl. No.: 12/242,631

(22) Filed: Sep. 30, 2008

(51) Int. Cl. B01J 21/06; B01J 23/78; B01J 27/25; B01J 23/00; C07C 5/367; B01J 23/32; B01J 21/18; B01J 23/10

(52) U.S. Cl. 585/444; 502/330; 502/201; 502/174; 502/304; 502/243; 502/307; 502/324

(57) ABSTRACT

A catalyst having at least 5 weight percent of an alumina compound useful for the dehydrogenation of alkylaromatic hydrocarbons to alkenylaromatic hydrocarbons and methods of use are disclosed.

### CROSS-REFERENCE TO RELATED APPLICATIONS

Not applicable.

### FIELD

The present invention generally relates to catalysts used for the conversion of hydrocarbons.

### BACKGROUND

Catalytic dehydrogenation of hydrocarbons using various catalyst compositions are well known in the art. In the dehydrogenation of alkylaromatic hydrocarbons to alkenylaromatic hydrocarbons, such as ethylbenzene to styrene, catalysts that exhibit higher conversion, selectivity, and increased stability are in constant development.

The current industry standard for an ethylbenzene catalyst for styrene production is a bulk metal oxide catalyst with iron/potassium (Fe/K) active phases with one or more promoters, such as cerium. Other components may also be added to the dehydrogenation catalyst to provide further promotion, activation or stabilization.

Normal catalyst deactivation can tend to reduce the level of conversion, the level of selectivity, or both, each which can result in an undesirable loss of process efficiency. There can be various reasons for deactivation of dehydrogenation catalysts. These can include the plugging of catalyst surfaces, such as by coke or tars, which can be referred to as carbonization; the physical breakdown of the catalyst structure; and the loss of promoters, such as the physical loss of an alkali metal compound from the catalyst or the agglomeration of potassium within the catalyst. Depending upon the catalyst and the various operating parameters that are used, one or more of these mechanisms may apply.

The carbonization of catalyst surfaces can be treated by the steaming and heating of the catalyst, referred to as decoking, but these regenerative operations can lead to the physical breakdown of the catalyst structure. Potassium can be mobile at high temperature, especially with steam. In the steam decoking process potassium movement and loss can be a problem, which can be further compounded by any physical breakdown of the catalyst structure.

The catalyst life of dehydrogenation catalysts is often dictated by the pressure drop across a reactor. An increase in the pressure drop lowers both the yield and conversion to the desired product. Physical degradation of the catalyst typically increases the pressure drop across the reactor. For this reason, the physical integrity of the catalyst is of major importance. Dehydrogenation catalysts containing iron oxide can undergo substantial changes under process conditions that decrease their physical integrity. For example, in the dehydrogenation of ethylbenzene to styrene, the catalyst is subjected to contact with hydrogen and steam at high temperatures (for example, 500° C. to 700° C.) and, under these conditions, Fe₂O₃, the preferred source of iron for the production of styrene catalysts, can be reduced to Fe₃O₄. This reduction causes a transformation in the lattice structure of the iron oxide, resulting in catalyst structures with less physical integrity and are more susceptible to degradation by contact with water at temperatures below 100° C. This degradation by contact with water is characterized by the catalyst bodies (e.g., pellets or granules) becoming soft and/or swollen and/or cracked. The water that contacts the catalysts may be in the form of liquid or a wet gas, such as air with a high humidity. The term “high humidity” herein refers to a relative humidity above about 50%.

The activity of dehydrogenation catalysts diminishes over time. Eventually the catalyst will deactivate to the point at which it must be replaced or regenerated. This can be expensive due to the lost production during replacement and/or the expenses involved in regenerating the catalyst. Any increase in stability of the catalyst that would promote a longer catalyst life would enhance the economics of the process using the catalyst.

In view of the above, it would be desirable to increase the stability of the catalyst, which would promote a longer catalyst life, increase its resistance to degradation due to decoking operations, assist in keeping the pressure drop across the reactor to a minimum, and increase its ability to withstand a high humidity environment.

### SUMMARY

Embodiments of the present invention generally include a catalyst comprising 30 to 90 weight percent of an iron compound, 1 to 50 weight percent of an alkali metal compound, and at least 5 weight percent of an alumina compound. The iron compound can comprise iron oxide and can be a potassium ferrite.

The alumina compound can be selected from the group consisting of alumina, a metal modified alumina, and metal aluminates. The catalyst can comprise at least 10 weight percent of an alumina compound.

The alkali metal compound can be selected from the group consisting of an alkali metal oxide, nitrate, hydroxide, carbonate, bicarbonate, and combinations thereof, and can comprise a sodium or potassium compound. The alkali metal compound can be a potassium ferrite.

The catalyst can further include from 0.5 to 25.0 weight percent of a cerium compound. The catalyst can further include 0.1 ppm to 1000 ppm of a noble metal compound. The catalyst can further include from 0.1 weight percent to 10.0 weight percent of a source for at least one of the following elements selected from the group consisting of aluminum, silicon, zinc, manganese, cobalt, copper, vanadium and combinations thereof.

An embodiment of the invention is a method for the dehydrogenation of alkylaromatic hydrocarbons to alkenylaromatic hydrocarbons. The method includes providing a dehydrogenation catalyst comprised of 10 to 90 weight percent of an iron compound, 1 to 50 weight percent of an alkali metal compound, and at least 5 weight percent of an alumina compound to a dehydrogenation reactor. A hydrocarbon feedstock comprised of alkylaromatic hydrocarbons and steam is supplied to the dehydrogenation reactor. The hydrocarbon feedstock and steam are contacted with the dehydrogenation catalyst within the reactor under conditions effective to dehydrogenate at least a portion of said alkylaromatic hydrocarbons to produce alkenylaromatic hydrocarbons. A product of alkenylaromatic hydrocarbons is recovered from the dehydrogenation reactor.

The alkylaromatic hydrocarbons in the feedstock can include ethylbenzene and the alkenylaromatic hydrocarbons of the product can include styrene. The alumina compound in the dehydrogenation catalyst can be selected from the group consisting of alumina, a metal modified alumina, and metal aluminates. The iron compound can be iron oxide and the alkali metal compound can be a potassium compound. The dehydrogenation catalyst can further comprise potassium ferrite. The dehydrogenation catalyst can include 0.5 to 25.0 weight percent of a cerium compound.

### BRIEF DESCRIPTION OF DRAWINGS

FIG. 1 is a graph of Styrene Selectivity versus EB Conversion for EB to styrene conversions using the catalyst produced in Batch 2.

FIG. 2 is a graph of Styrene Selectivity versus EB Conversion for for EB to styrene conversions using the catalyst produced in Batch 5.

### DETAILED DESCRIPTION

To achieve higher performance, longer run times, and lower steam to hydrocarbon ratios, efforts have been made to develop a catalyst with improved physical properties. The approach of the current invention involves the addition of a support material, such as alumina, metal modified aluminas or metal modified aluminates, to a traditional mixed metal oxide formula to stabilize the active species and improve the physical properties. A series of catalysts have been prepared that contain approximately 25% alumina along with Fe/K/Ce ingredients. Catalysts with good surface area and porosity have been prepared using this approach. X-ray diffraction data shows that potassium ferrite phases have been formed from the iron oxide starting material. Ferrite phases are generally considered active species for dehydrogenation reactions. The alumina addition has been observed to promote the formation of ferrite phases in these catalyst formulations.

Embodiments of the present invention generally include a catalyst comprising 30 to 90 weight percent of an iron compound, 1 to 50 weight percent of an alkali metal compound, and at least 5 weight percent of an alumina compound. The iron compound can comprise iron oxide and can be a potassium ferrite. The alumina compound can be selected from the group consisting of alumina, a metal modified alumina, and metal aluminates.

The alkali metal compound can be selected from the group consisting of an alkali metal oxide, nitrate, hydroxide, carbonate, bicarbonate, and combinations thereof, and can comprise a sodium or potassium compound. The alkali metal compound can be a potassium ferrite.

The catalyst can further include from 0.5 to 25.0 weight percent of a cerium compound. The catalyst can further include 0.1 ppm to 1000 ppm of a noble metal compound. The catalyst can further include from 0.1 weight percent to 10.0 weight percent of a source for at least one of the following elements selected from the group consisting of aluminum, silicon, zinc, manganese, cobalt, copper, vanadium and combinations thereof.

An embodiment of the invention is a method for the dehydrogenation of alkylaromatic hydrocarbons to alkenylaromatic hydrocarbons. The method includes providing a dehydrogenation catalyst comprised of 10 to 90 weight percent of an iron compound, 1 to 50 weight percent of an alkali metal compound, and at least 5 weight percent of an alumina compound to a dehydrogenation reactor. A hydrocarbon feedstock comprised of alkylaromatic hydrocarbons and steam is supplied to the dehydrogenation reactor. The hydrocarbon feedstock and steam are contacted with the dehydrogenation catalyst within the reactor under conditions effective to dehydrogenate at least a portion of said alkylaromatic hydrocarbons to produce alkenylaromatic hydrocarbons. A product of alkenylaromatic hydrocarbons is recovered from the dehydrogenation reactor.

The alkylaromatic hydrocarbons in the feedstock can include ethylbenzene and the alkenylaromatic hydrocarbons of the product can include styrene. The alumina compound in the dehydrogenation catalyst can be selected from the group consisting of alumina, a metal modified alumina, and metal aluminates. The iron compound can be iron oxide and the alkali metal compound can be a potassium compound. The dehydrogenation catalyst can further comprise potassium ferrite. The dehydrogenation catalyst can include 0.5 to 25.0 weight percent of a cerium compound.

Small changes in surface area, porosity, and pore diameter can have a significant impact on bulk mixed metal oxide styrene catalysts. For example, a larger pore diameter and an increased stability of potassium can reduce the need for decoking of the catalyst. A reduction in the need for decoking operation can lessen potassium mobilization and loss. Reduced decoking can also reduce the demand for steam into the system, thus reducing energy costs.

The order of addition and the type of reagents used, whether it is the metal oxide or the pore forming agents, can significantly affect these physical properties. Catalyst Batches 1 and 2 were prepared by addition of potassium as a final step. The next series, Batches 3 and 4, explored an alternative method utilizing a single step preparation that included the potassium compound. Batch 5 substituted magnesium aluminum oxide for aluminum oxide. The presence of a green color in the finished catalyst, a result of the potassium monoferrite phase from the interaction of K and Fe, was not inhibited in these preparations.

Two different options were explored for the iron oxide starting material. The traditionally-used red iron oxide, Fe₂O₃, is one substrate that was used in Batch 1 and Batch 3, and yellow iron oxide, FeO(OH), was used in Batches 2, 4, and 5. The yellow iron oxide tends to form smaller crystallites after calcination and reacts more readily with other inorganic substrates. For test Batch 1 red iron oxide synthetic hematite was used and for test Batch 2 yellow iron oxide lepidocrocite was used. Synthetic hematite produced by calcination of synthetic goethite is often used to catalyze the conversion of ethylbenzene to styrene because these materials often have the highest purity (>98% Fe₂O₃). Other iron oxides, although not tested in this experiment, may also be used in accordance with the invention can include, but are not limited to: black iron oxides such as magnetite, brown iron oxides such as maghemite, and other yellow iron oxides such as goethite. The 1-5 micron alumina that was tested in Batches 2 and 4 has a surface area of 2.7 m²/g.

### EXPERIMENTAL EXAMPLES

### Batches 1 & 2—Examples of Multi Step Preparation

In the multi step process small batches of approximately 100 g of catalyst material were prepared by hand. Ingredients were mixed and DI water was added to form a paste that was suitable for forming into pellets or tiles using a Carver Hydraulic Press. The ingredient list is shown in Table 1. The catalysts have the same molar proportions of Fe, K, Ce, Al, Ca, and Mo components. The same amount of cement, added for strength, was used in each. Also, graphite, methyl cellulose, and stearic acid were added as extrusion aids and pore formers.

TABLE 1
Ingredient list with Descriptions and Lot Numbers.

| Chemical | Description | Source |
|---|---|---|
| Fe₂O₃ | Red iron oxide | Bailey PVS |
| FeO(OH) | Yellow Iron Oxide | Strem Lot# B5327066 |
| K₂CO₃ | Potassium carbonate, | AlfaAesar, LOT# L12Q045 |
| | ACS 99.0% min | |
| CaCO₃ | Calcium carbonate, 98% | Strem, LOT# B2789046 |
| Ce₂(CO₃)₃•5H₂O | Cerium oxide | Tianjiao, LOT# 20060701 |
| Al₂O₃ | Aluminum oxide, 1-5 | Strem, LOT# B9139096 |
| | micron powder, 99+% | |
| Al₂O₃ | Aluminum oxide, fused, | Sigma-Aldrich, BATCH# |
| | .325 mesh + 10 micron, | 01728TD |
| | 99+% | |
| MoO₃ | Molybdenum oxide, | AlfaAesar, LOT# C29Q06 |
| | ACS, 99.5% min | |
| Methyl cellulose | Methyl cellulose, 25 cP | Sigma-Aldrich, BATCH# |
| | | 095K0189 |
| C₁₈H₃₆O₂ | Stearic acid | Sigma-Aldrich, BATCH# |
| | | 08601PD |
| C(s) | Graphite, powder, <20 | Sigma-Aldrich, BATCH# |
| | micron synthetic | 04430TC |
| Calcium | Lumnite cement | Heidelberger, LOT# 0514 |
| aluminate cement | | |

After forming, the catalysts were aged overnight in a sealed container from 20° C. to 30° C., and then dried at 115° C. Next, the catalysts were calcined with a maximum temperature of 775° C. and held for 4 hours. A more detailed description of Batches 1 and 2 follow.

Batch 1 was prepared by dry mixing red iron oxide (36 g), cerium carbonate (11 g), calcium carbonate (6 g), aluminum oxide 1-5 micron (23 g), molybdenum oxide (1 g), methyl cellulose—25 cP (0.5 g), stearic acid (0.75 g), graphite (0.75 g) and cement (4 g). The formulation spreadsheet is shown in Table 2. These reagents were added together and well mixed. Enough deionized water was added until the mixture was wet enough to form large clumps. Then, potassium carbonate (19 g) was added and the mixture was allowed to react and to thicken. Approximately 2 grams of prepared catalyst was put in a 13 mm die and 4,000-5,000 psig was applied to make a pellet. Ten to fifteen pellets were made at one time and placed in a ceramic dish to dry overnight. The remaining catalyst was placed in a zip-top plastic bag and hand-pressed until flat. A ceramic dish was weighed and the weight was recorded. Then, approximately 10 grams of hand-pressed catalyst was added to the ceramic dish and the weight was recorded. The remaining hand-pressed catalyst was then broken into pieces and placed in a ceramic dish to dry overnight. After approximately 24 hours, the catalyst was placed in an oven and dried at 115° C. for approximately 2 hours. The catalyst was then weighed and the weight recorded. Then, the dried catalyst was calcined according to the following ramping procedure: 350° C. for 1 hour, 600° C. for 1 hour and then ramped to 775° C. at a rate of 10° C./min and held for 4 hours. Once this cycle was completed the oven returned to 115° C. until the catalyst was removed. The calcined catalyst was weighed and the weight recorded.

TABLE 2
The formulation spreadsheet for Batch 1 with starting material
weight percent, calcined mole percent and calcined weight percent.
SemSup 1: red iron oxide with 1-5 micron alumina

| | | ingredient | | | | calcined | calcined | calcined | calcined | calcined |
|---|---|---|---|---|---|---|---|---|---|---|
| ingredient | wt grams | wt % | MW g/mol | stoich | moles | MW g/mol | stoich | wt grams | mol % | wt % |
| Fe2O3 red Bailey as recvd | 36 | 35.29 | 159.7 | 2 | 0.451 | 159.7 | 2 | 36 | 35.12 | 38.51 |
| K2CO3 | 19 | 18.63 | 138.2 | 2 | 0.275 | 138.2 | 2 | 19 | 21.42 | 20.38 |
| CaCO3 | 6 | 5.88 | 100.1 | 1 | 0.060 | 56.1 | 1 | 3.4 | 4.67 | 3.61 |
| Ce2(CO3)3-5 H2O Tianjiao | 11 | 10.78 | 550.2 | 2 | 0.040 | 172.1 | 1 | 6.9 | 3.11 | 7.38 |
| Al2O3 1-5 micron | 23 | 22.55 | 102 | 2 | 0.451 | 102 | 2 | 23 | 35.13 | 24.67 |
| MoO3 | 1 | 0.98 | 143.9 | 1 | 0.007 | 143.9 | 1 | 1 | 0.54 | 1.07 |
| methyl cellulose 25 cP | 0.5 | 0.49 | 1 | 0 | 0.000 | 0 | 1 | 0 | 0.00 | 0.00 |
| stearic acid | 0.75 | 0.74 | 1 | 0 | 0.000 | 0 | 1 | 0 | 0.00 | 0.00 |
| graphite | 0.75 | 0.74 | 12 | 0 | 0.000 | 0 | 1 | 0 | 0.00 | 0.00 |
| Cement | 4 | 3.92 | | | | | | 4 | 0.00 | 4.29 |
| | 102 | 100.00 | | | 1.284 | | | 93.24 | 100.00 | 100.00 |

Batch 2 was prepared in the same manner as Batch 1 except that yellow iron oxide (40 g) was substituted equimolar for the red iron oxide.

The amount of water added during preparation was recorded for each preparation. Also the appearance of the catalysts after drying and after calcining was recorded. These observations are shown in Table 3 for batches 1 and 2.

TABLE 3
Observations during catalyst synthesis and water addition amounts
for batches 1 and 2.

| | Observations After | | |
|---|---|---|---|
| | Drying at 115° C./ | Observations After Calcining | Water |
| Catalyst | 2 hours | at 775° C./4 hours | Added |
| Batch 1 | no change in color, | dark brown catalyst with a | 17.37 g |
| | white frosted spots | greenish tint, tan frosting and | |
| | | a few white spots | |
| Batch 2 | yellow-brown catalyst | brown catalyst with a green | 25.49 g |
| | | tint | |

All catalysts had high crush strengths (qualitative) after the calcinations were performed. Hand made pellets were tested and had crush strengths greater than 60 psi.

BET surface area and Hg intrusion data was recorded for each catalyst. A summary is shown in Table 4.

TABLE 4
BET surface area and Hg intrusion data

| | | | Hg Intrusion | | | |
|---|---|---|---|---|---|---|
| | | | Pore | | Avg vs area | Avg (4V/A) |
| | | BET S.A. | Volume | Hg S.A. | Hg pore D | Hg pore D |
| Batch # | Catalyst description | m2/g | mL/g | m2/g | Angstom | Angstom |
| 1 | Red iron oxide - 1-5 micron alumina | 1.7 | 0.35 | 1.61 | 3197 | 8804 |
| 2 | Yellow iron oxide - 1-5 micron | 2.9 | 0.53 | 3.09 | 2248 | 6823 |
| | alumina | | | | | |

The aim of the first round of catalyst preparations was to determine the feasibility of a Fe/K/Ce dehydrogenation catalyst that has 25 wt % alumina and whether the alumina will allow the formation of ferrite phases. The calcined catalyst should have a final surface area of 1-4 m²/g, porosity greater than 0.1 mL/g, and acceptable crush strength, such as greater than 60 psi.

The potassium carbonate was added to the other ingredients only after they were mixed and wetted in both Batch 1 and 2. The basic potassium carbonate reacts with the acidic iron oxide and the order of how the acidic and basic ingredients are mixed can be important.

The BET surface area data were conducted with nitrogen and are shown in Table 4. The values are in an acceptable range for styrene catalysts.

Table 4 also shows the Hg intrusion data. The values were obtained from crushed 13 mm pellets, so the data can be useful, but not necessarily the exact value for a commercial-grade extrudate. A catalyst with large pores (more than 0.1 micron) and high porosity (greater than 0.2 mL/g) can show improved performance due to reduced diffusional constraints. The Hg intrusion data in Table 4 shows that these initial catalyst formulations do show high porosity (pore volume) and have large average pore diameters (versus area).

The x-ray diffraction (XRD) data of Batches 1 and 2 indicated that the formulations were fairly similar. Aluminum oxide and cerium oxide were prominent but not iron oxide. The iron was observed as monoferrite (KFeO₂), a lower polyferrite (K₂Fe₄O₇) or an alkali/aluminum/iron mixed oxide. Batch 1 showed significant monoferrite and polyferrite phases. Batch 2 was similar to batch 1 except the monoferrite concentration was lower and the polyferrite higher.

### Batches 3 and 4—Examples of a Single Step Preparation

The same ingredient ratios were used in all of the Batch formulations as given herein. The weight percentages after calcination and assuming the highest valent oxide for each ingredient gave the following: iron oxide (38.6%), potassium carbonate (20.4%), calcium oxide (3.6%), cerium oxide (7.4%), aluminum oxide (24.7%), molybdenum oxide (1.07%) and calcium aluminate cement (4.3%). The ingredient list is shown in Table 1.

Batch 3 was prepared by dry mixing red iron oxide (36 g), cerium carbonate (11 g), potassium carbonate (19 g), calcium carbonate (6 g), aluminum oxide (1-5 micron, 23 g), molybdenum oxide (1 g), methyl cellulose—25 cP (0.5 g), stearic acid (0.75 g), graphite (0.75 g) and cement (4 g). These reagents were added together and well mixed. Deionized water was added and the mixture was allowed to react and to thicken. Approximately 2 grams of prepared catalyst was added to a 13 mm die and 4,000-5,000 PSI was applied to make a pellet. Ten pellets and one 2.5 cm×2.5 cm tile were made at one time and placed in a ceramic dish to dry overnight at from 20° C. and 30° C. The remaining catalyst was placed in a zip-top plastic bag and hand-pressed until flat. A ceramic dish was weighed and the weight recorded. Then, approximately 10 grams of hand-pressed catalyst was added to the ceramic dish and the weight recorded. The remaining hand-pressed catalyst was then broken into pieces and placed in a ceramic dish to dry overnight. After approximately 24 hours, the catalyst was placed in an oven and dried at 115° C. for approximately 2 hours. The catalyst was then weighed and the weight recorded. Then, the dried catalyst was calcined according to the following ramping procedure: 350° C. for 1 hour, 600° C. for 1 hour and then ramped to 775° C. at a rate of 10° C./min and then held for 4 hours. Once this cycle was completed the oven returned to 115° C. and held until the catalyst was removed. The calcined catalyst was weighed and the weight recorded.

Batch 4 was prepared in the same manner as Batch 3 except that yellow iron oxide (40 g) was substituted equimolar for the red iron oxide.

All catalysts seemed qualitative to have good crush strengths after the calcinations were performed. The catalysts were analyzed for BET surface area and Hg Intrusion. Hand made pellets were tested and had crush strengths greater than 60 psi.

### Observations and Results

Catalysts in Batches 1 and 2 were prepared by wet mixing all the ingredients except the potassium carbonate, which is added separately at the end of the mixing steps. For Batches 3 and 4 the potassium carbonate was added along with the other ingredients in the mixing step.

The amount of water added during preparation was recorded for each preparation. Also the appearance of the catalysts after drying and after calcining was recorded. These observations are shown in Table 5 for Batches 3 and 4.

TABLE 5
Qualitative Observations During Catalyst Preparation

| | Observations | Oservations | |
|---|---|---|---|
| | After Drying at | After Calcining at | Water |
| Catayst | 115° C./2 hours | 775° C./4 hours | Added |
| Batch 3 | No color change, a | Brown, dark brown patches, | 13.18 g |
| | few white frosted spots | few white frosted spots | |
| Batch 4 | No change in color | Brown catalyst | 21.94 g |

The resulting catalyst color formed with these alternative preparation methods had less green tints and more brown coloration than the initial formulations that had the potassium addition as the last step. Batches 1 and 2 showed greenish tint due to the formation of potassium monoferrite. The brown color generally indicates the presence of polyferrite phases that have a higher Fe to K content. The frosting that was observed is likely due to free potassium carbonate at the surface.

TABLE 6
The physical property data for Batches 3 and 4 catalysts

| | | BET SA | Hg pore | Hg SA | Hg pore D |
|---|---|---|---|---|---|
| Batch # | Catalyst description | m2/g | vol mL/g | m2/g | A* area |
| 3 | Single step version of batch 1 | 1.7 | 0.31 | 1.80 | 2993 |
| | Red iron oxide - 1-5 micron alumina | | | | |
| 4 | Single step version of batch 2 | 2.7 | 0.41 | 3.07 | 1962 |
| | Yellow iron oxide - 1-5 micron alumina | | | | |

The BET surface area and the pore volume and diameter by Hg intrusion are important physical property values for styrene catalysts. The data for Batches 3 and 4 are shown in Table 6. The BET surface areas are desirably low at 1-3 m²/g. The yellow iron oxide formulations tend to show a slightly higher surface area. The calcined catalyst should have a final surface area of 1-4 m²/g, porosity greater than 0.1 mL/g, and acceptable crush strength, such as greater than 60 psi.

The Batch 3 and 4 formulations were single step versions of Batches 1 and 2. Red iron oxide was used for batches 1 and 3 and yellow iron oxide for Batches 2 and 4. The single step procedure produced a catalyst with slightly lower pore volume when red iron oxide was used but no significant differences for the yellow iron oxide batches.

Batch 5—Example of Catalyst Including Magnesium Aluminum Oxide (Same as Batch 2 with Aluminum Oxide Substituted with Magnesium Aluminum Oxide)

Batch 5 was prepared by dry mixing yellow iron oxide, cerium carbonate, calcium carbonate, magnesium aluminum oxide, molybdenum oxide, methyl cellulose (25 cP), graphite, and cement. These reagents were added to a mix muller and mulled for 2 hours. Enough deionized water was added until the mixture formed large clumps. Then, potassium carbonate was added and the mulled mixture was allowed to react and mull until well mixed. The mulled mixture was transferred to the extruder and was extruded under 3 metric tons of pressure. The extrudates were placed in a plastic bag and allowed to cure overnight at from 20° C. and 30° C. After approximately 24 hours, the catalyst was placed in an oven and dried at 115° C. for approximately 24 hours. Then, the dried catalyst was calcined according to the following ramping procedure: 350° C. for 1 hour, 600° C. for 1 hour and then ramped to 775° C. at a rate of 10° C./min and then held for 4 hours. Once this cycle was completed the oven returned to 115° C. and was held until the catalyst was removed.

The prepared catalyst was analyzed for BET surface area and for pore volume and diameter. The following Table 7 shows the data obtained for the Batch 5 catalyst.

| | | | Sample | Hg pore | | Hg pore | | Water |
|---|---|---|---|---|---|---|---|---|
| | Catalyst | | weight | vol | Hg SA | D A* | Hg pore D | added |
| Batch # | descriptor | SA m2/g | (g) | mL/g | m2/g | area | A* (4V/A) | weight |
| 5 | CoMO4MX | 2.0054 | 1.6059 | 0.2804 | 3.736 | 2028 | 3002 | 191.86 |

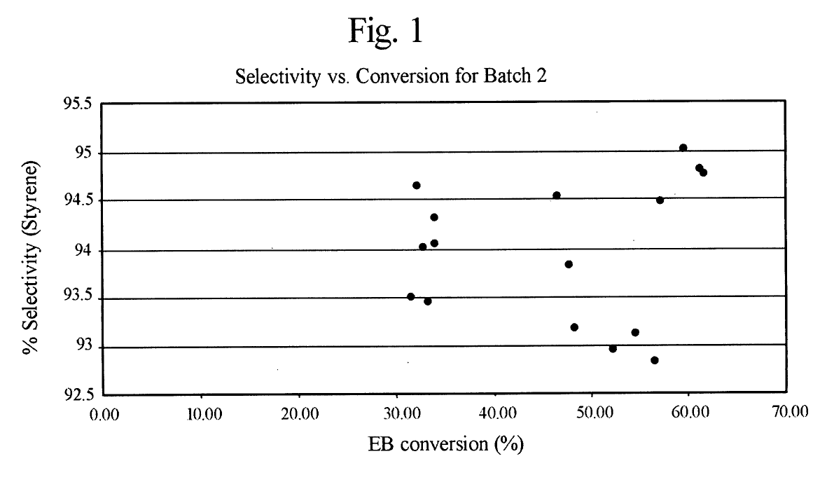

The catalyst produced from Batch 2 prepared with yellow iron oxide and aluminum oxide was analyzed in an isothermal bench scale reactor for ethylbenzene dehydrogenation to styrene at various reactor conditions. Steam to ethylbenzene ratios ranged between 7 to 9 and temperatures from 590° C. and 630° C. The LHSV was held at 3 hr⁻¹ and the partial pressure of EB/H₂O was 700. The reactor pressure was set at 1350 mbar. FIG. 1 is a graph of Styrene Selectivity versus EB Conversion for EB to styrene conversions using the catalyst produced in Batch 2. The data from FIG. 1 shows that the Batch 2 catalyst can be used in the dehydrogenation of ethylbenzene to styrene.

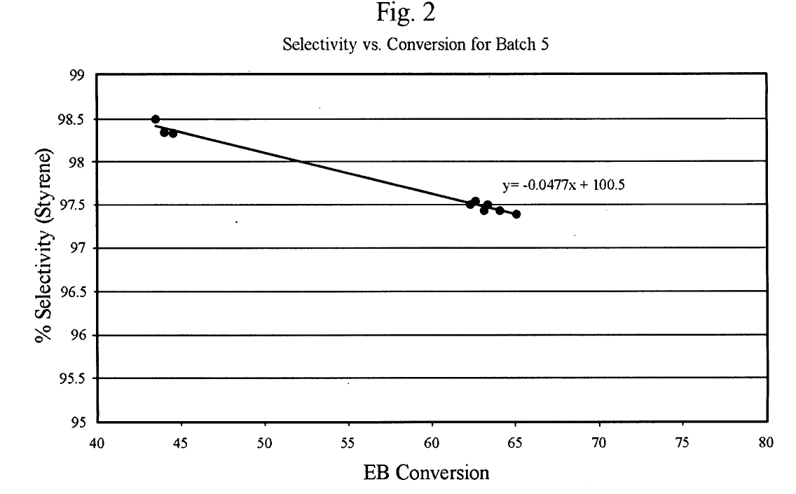

The catalyst produced from Batch 5 prepared with yellow iron oxide and magnesium aluminum oxide was analyzed in an isothermal bench scale reactor for ethylbenzene dehydrogenation to styrene at various reactor conditions. Steam to ethylbenzene ratios ranged between 7 to 9 and temperatures from 590° C. and 630° C. The LHSV was held at 3 hr⁻¹ and the partial pressure of EB/H₂O was 700. The reactor pressure was set at 1350 mbar. FIG. 2 is a graph of Styrene Selectivity versus EB Conversion for EB to styrene conversions using the catalyst produced in Batch 5. The data from FIG. 2 shows that the Batch 5 catalyst can be used in the dehydrogenation of ethylbenzene to styrene.

Alumina compounds can be added to a dehydrogenation catalyst composition in significant quantities to enhance the strength and durability of the catalyst. These materials can interact with the iron and potassium to inhibit sintering and reduction of the iron oxide and can stabilize the potassium and slow its migration. The alumina compound can be selected from the group consisting of alumina, metal modified alumina, and metal aluminates or combinations thereof. The alumina compound content in the catalyst can be at least 5 wt % and can range up to 10 wt %, 20 wt %, 40 wt %, 60 wt % or 80 wt % of the finished catalyst.

Metal modified alumina compounds can include alumina modified with a metal or metal oxide. They can include a physical mixture of oxides, carbonates, nitrates, hydroxides, bicarbonate, and combinations thereof or other compounds; co-precipitated mixtures; incipient wetness additions; and chemical vapor depositions as non-limiting examples.

The metals can include as non-limiting examples: alkali metals; alkaline earths; lanthanides; transition metals; Ga; In; Ge; Sn; Pb; As; Sb; Bi; and combinations of the above with alumina. Metal aluminates can include, as non-limiting examples, mixed metal oxides of alumina including beta alumina; spinels; perovskites; and combinations thereof.

Further non-limiting examples include various compositions and molar ratios of the following: Al₂O₃; MgAlO₄; Mg/Al; Li/Al; Na/Al; K/Al; Fe/K/Al; Al—K₂CO₃; Al2O₃/Al(OH)₃; Mn—Al oxide; Na—Mn—Al oxide; K—Mn—Al oxide; Al—CuO; Al—ZnO; and combinations thereof.

The components can be calcined at an elevated temperature prior to being used as ingredients in the various compositions.

The term “activity” refers to the weight of product produced per weight of the catalyst used in a process per hour of reaction at a standard set of conditions (e.g., grams product/gram catalyst/hr).

The term “alkyl” refers to a functional group or side-chain that consists solely of single-bonded carbon and hydrogen atoms, for example a methyl or ethyl group.

The term “deactivated catalyst” refers to a catalyst that has lost enough catalyst activity to no longer be efficient in a specified process. Such efficiency is determined by individual process parameters.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A catalyst comprising:
30 to 90 weight percent of an iron compound;
1 to 50 weight percent of an alkali metal compound; and
at least 5 weight percent of an alumina compound.

2. The catalyst of claim 1, wherein the iron compound comprises iron oxide.

3. The catalyst of claim 1, wherein the iron compound comprises a potassium ferrite.

4. The catalyst of claim 1, wherein the alkali metal compound is selected from the group consisting of an alkali metal oxide, nitrate, hydroxide, carbonate, bicarbonate, and combinations thereof.

5. The catalyst of claim 1, wherein the alkali metal compound comprises a sodium or potassium compound.

6. The catalyst of claim 1, wherein the alkali metal compound comprises a potassium ferrite.

7. The catalyst of claim 1, wherein the alumina compound is selected from the group consisting of alumina, a metal modified alumina, and metal aluminates.

8. The catalyst of claim 1, comprising at least 10 weight percent of an alumina compound.

9. The catalyst of claim 1, comprising at least 20 weight percent of an alumina compound.

10. The catalyst of claim 1, further comprising from 0.5 to 25.0 weight percent of a cerium compound.

11. The catalyst of claim 1, further comprising 0.1 ppm to 1000 ppm of a noble metal compound.

12. The catalyst of claim 1, further comprising from 0.1 weight percent to 10.0 weight percent of a source for at least one of the following elements selected from the group consisting of aluminum, silicon, zinc, manganese, cobalt, copper, vanadium and combinations thereof.

13. A non-oxidative dehydrogenation catalyst for dehydrogenating a hydrocarbon feed stream in a hydrocarbon reaction zone, wherein the components of the hydrocarbon feed stream in the reaction zone consist essentially of an alkylaromatic hydrocarbon and steam, comprising:
10 to 90 weight percent iron oxide;
1.0 to 50 weight percent of a potassium compound;
from 0.5 to 12.0 weight percent of a cerium compound; and
at least 5 weight percent of an alumina compound.

14. The catalyst of claim 13, wherein the alumina compound is selected from the group consisting of alumina, a metal modified alumina, and metal aluminates.

15. A method for the dehydrogenation of alkylaromatic hydrocarbons to alkenylaromatic hydrocarbons comprising:
providing a dehydrogenation catalyst comprised of 10 to 90 weight percent of an iron compound, 1 to 50 weight percent of an alkali metal compound, and at least 5 weight percent of an alumina compound to a dehydrogenation reactor;
supplying a hydrocarbon feedstock comprised of alkylaromatic hydrocarbons and steam to the dehydrogenation reactor;
contacting the hydrocarbon feedstock and steam with the dehydrogenation catalyst within the reactor under conditions effective to dehydrogenate at least a portion of said alkylaromatic hydrocarbons to produce alkenylaromatic hydrocarbons; and
recovering a product of alkenylaromatic hydrocarbons from the dehydrogenation reactor.

16. The method of claim 15, wherein the alkylaromatic hydrocarbons in the feedstock includes ethylbenzene and the alkenylaromatic hydrocarbons of the product includes styrene.

17. The method of claim 15, wherein the alumina compound in the dehydrogenation catalyst is selected from the group consisting of alumina, a metal modified alumina, and metal aluminates.

18. The method of claim 15, wherein the iron compound is iron oxide.

19. The method of claim 15, wherein the alkali metal compound is a potassium compound.

20. The method of claim 15, wherein the dehydrogenation catalyst further comprises potassium ferrite.

21. The method of claim 15, wherein the dehydrogenation catalyst further comprises 0.5 to 25.0 weight percent of a cerium compound.
